(19) United States
(12) Patent Application Publication
(10) Pub. No.: US 2022/0349870 A1
(43) Pub. Date: Nov. 3, 2022

(54) Method for Detecting American Foulbrood in a Beehive

(71) Applicant: CRC For Honey Bee Products, an Australian Company of UMA, Perth, Western Australia (AU)

(72) Inventors: Julia Grassl, Carine, Western Australia (AU); Jessica M. Moran, North Perth (AU)

(21) Appl. No.: 17/642,594

(22) PCT Filed: Oct. 1, 2020
(86) PCT No.: PCT/AU2020/051048

§ 371 Date: Mar. 11, 2022

(30) Foreign Application Priority Data
Oct. 3, 2019 (AU) 2019903716
Jul. 3, 2020 (AU) 2020902277

(51) Int. Cl. G01N 33/497; G01N 33/00

(52) U.S. Cl. CPC G01N 33/497; G01N 33/0047; G01N 2033/4977

(57) ABSTRACT

The present invention provides a method for the detection of American foulbrood (AFB) in a beehive, said method comprising the steps of taking a sample from the beehive and testing the sample for a range of compounds, wherein the presence of one or more of the compounds in a relative amount of at least three times that of a non-infected hive indicates the presence of AFB in the beehive.

### TECHNICAL FIELD

The present invention relates to a non-invasive method for detecting American Foulbrood in beehives.

### BACKGROUND ART

Honey bee (Apis mellifera) health is critical for world-wide sustainability of honey bee pollination services that contribute to global food security. American foulbrood (AFB), caused by Paenibacillus larvae, is the most devastating bacterial disease of honey bees. Infected larvae reduce to a glue-like mass, usually accompanied by a foul odour.

Early detection of AFB is crucial, as infections can quickly reach critical levels and spread to surrounding beehives. Infected hives and equipment require burning in many countries (such as Australia), where antibiotic treatment is prohibited. This disease can result in substantial economic losses to beekeepers. Current AFB field diagnostics are limited as they rely on beekeepers checking each frame within a hive to first identify symptoms. The process of opening and inspecting hives can spread disease via equipment and is incredibly time-consuming, particularly for beekeepers who manage more than 100 hives.

There is an antibody-based diagnostic test for AFB (similar in construction and use to a pregnancy test) from VITA Bee Health, which reacts specifically to antibodies associated with the pathogen Paenibacillus larvae. A small sample of infected larvae is smeared on the target and allowed to react. However, this test requires the opening of hives and the identification and sampling of infected brood cells. This renders it impractical for the testing of large apiaries and requires initial visual identification of a potentially affected cell.

Sniffer dogs trained to detect AFB-diseased hives in apiaries have not been widely adopted due to time, effort and cost associated with dog training, and the poor working conditions for the animal (for example, bee stings and heat if wearing a protective suit).

There is a need to provide a new method for detection of AFB; or at least the provision of an alternative to compliment the previously known detection methods. The present invention seeks to provide an improved or alternative method for detection of AFB.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a method for the detection of American foulbrood (AFB) in a beehive, said method comprising the step of:
a) taking a sample from the beehive;
b) testing the sample for compounds from the list comprising:
1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS #14667-55-1); Propanamide, 2-methyl-(CAS # 563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS #112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS #590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ 016 alkene; AFB NIL 9; Furan, 3-methyl-(CAS #930-27-8)
   wherein the presence of one or more of the compounds in a relative amount of at least three times that of a non-infected hive indicates the presence of AFB in the beehive.

The method represents the identification of compounds released by Paenibacillus larvae infected larval honey bees.

Preferably the compounds are chosen from the list comprising: 2(3H)-Furanone, dihydro-3-hydroxy-4,4-dimethyl-(CAS #52126-90-6); 3-Buten-1-ol, 3-methyl-(CAS #763-32-6); Acetamide (CAS #60-35-5); Benzeneacetamide (CAS #103-81-1); Butanamide (CAS #541-35-5); Butanamide, 3-methyl-(CAS #541-46-8); Hexanamide (CAS #628-02-4); Pentanoic acid, 3-methyl-2-oxo-, methyl ester (CAS #3682-42-6); Propanamide, 2-methyl-(CAS #563-83-7); Pyrazine, 2,5-dimethyl-(CAS #123-32-0); AFB NIL 1; AFB NIL 2; AFB NIL 6; AFB NIL 7.

Preferably the sample is taken by a non-invasive method that does not disturb the bees in the hive. Preferably the sample is a sample of air from the interior of the beehive. Preferably the detection means is a handheld, mobile, portable or point-of-use diagnostic tool.

Preferably the compounds are volatile organic compounds (VOCs).

The present invention further provides a kit for detecting the presence of American foulbrood (AFB) in a beehive, said kit comprising:
i.) a means for testing the sample for compounds from the list comprising:
   1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8), Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS # 123-32-0); Pyrazine, trimethyl-(CAS #14667-55-1); Propanamide, 2-methyl-(CAS # 563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS # 112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS # 590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ 016 alkene; AFB NIL 9; Furan, 3-methyl-(CAS #930-27-8); and
ii.) instructions for use
   wherein the presence of one or more of the compounds in a relative amount of at least three times that of a non-infected hive indicates the presence of AFB in the beehive.

The present invention further provides a device for detecting the presence of American foulbrood (AFB) in a beehive, said device comprising:
i.) a means for testing the sample for compounds from the list comprising:
   1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8), Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS # 123-32-0); Pyrazine, trimethyl-(CAS #14667-55-1); Propanamide, 2-methyl-(CAS # 563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS # 112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS # 590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ 016 alkene; AFB NIL 9; Furan, 3-methyl-(CAS #930-27-8)
      wherein the presence of one or more of the compounds in a relative amount of at least three times that of a non-infected hive indicates the presence of AFB in the beehive.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a Principal component analysis (PCA) of volatile compound profiles of honey bee larvae. The first two principal components (PCs) are displayed, together representing 64% of the total variation. Each point represents a single sample's volatile profile (accumulated from 3 replicate measurements). PC1 separates samples based on AFB infection status, whereas PC2 separates apiaries. Colour represents AFB infection status (red=AFB diseased, black=healthy, yellow=non-symptomatic larvae taken from diseased frames), while shape represents apiary origin of the sample. The percentage of variation in volatile profile explained by each PC is shown on the axes.

FIG. 2 is a heatmap for the hierarchical clustering of volatile profiles. Rows correspond to differentially abundant volatile compounds, and columns represent averages of replicate measurements of each sample. Data were log10 transformed and scaled. Greater peak intensities (abundances) of individual compounds are represented by black, lower intensities in grey, and white where the peak was not detected. Clustering of samples shows two main branches, which correspond with healthy larvae (left) and AFB-diseased larvae (right).

FIG. 3 is an example section of the total ion chromatogram at 33.4-36 min, displaying compounds present in an AFB-infected brood sample and healthy brood.

FIG. 4 is an example section of the total ion chromatogram at 16-18.3 min, displaying compounds present in an AFB-infected brood sample and healthy brood.

FIG. 5 is a spectra for the compound identified as AFB NIL 1.

FIG. 6 is a spectra for the compound identified as AFB NIL 2.

FIG. 7 is a spectra for the compound identified as AFB NIL 6.

FIG. 8 is a spectra for the compound identified as AFB NIL 7.

FIG. 9 is a spectra for the compound identified as AFB NIL 8.

FIG. 10 is a spectra for the compound identified as AFB NIL 9.

FIG. 11 is a spectra for the compound identified as 4-Hexen-3-ol.

FIG. 12 is a spectra for the compound identified as 2-dodecanone.

FIG. 13 is a spectra for the compound identified as 2-tetradecanone.

FIG. 14 is a spectra for the compound identified as pentanoic acid, 3-methyl-2-oxo-, methyl ester.

### DESCRIPTION OF INVENTION

### Detailed Description of the Invention

The present inventors have discovered a range of volatile organic compounds (VOCs) that are diagnostic of infection with Paenibacillus larvae bacteria, causing American Foulbrood (AFB), in honey bee larvae. The compounds discovered may be statistically more abundant in hives infected with AFB than uninfected hives, or the compounds may be only present in samples that have infections and are absent from samples from uninfected hives.

Without being bound by theory, certain VOCs are believed to be associated with AFB, and therefore may be used to detect viable, recently viable, growing bacteria, or bacteria in spore form in bee larvae. Although several VOCs have been predicted to be associated with AFB, no reliable and specific test has yet been developed (Gochnauer & Shearer (1981) J Apicultural Research, 20(2):104-109; Gochnauer (1981) J Apicultural Research, 20(2):110-114; Gochnauer, & Margetts (1982) J Apicultural Research, 21(3):161-164).

Accordingly, in various embodiments, the present invention is directed to detecting one or more VOCs that are associated with the metabolism, presence, and/or growth of AFB in infected larvae in order to detect the presence or absence, and/or early infection with the bacteria. The terms “unhealthy hive” and “infected hive” are used interchangeably in the present invention and refer to hives that have Paenibacillus larvae infections present, causing American Foulbrood (AFB). The terms “healthy hive” and “uninfected hive” are used interchangeably in the present invention and refer to hives that do not have Paenibacillus larvae bacteria present.

Experimental results have shown approximately 37 VOCs (derivatives of alcohols, aldehydes, alkenes, amides, heteroaromatic compounds, esters, ketones, lactones, lactams, and sulfurous compounds) that are present in statistically different amounts in samples positive for AFB infection compared to samples negative for AFB infection by PCR testing. Preferably, the compounds are present in an AFB infected hive in amounts of at least three times that present in a non-infected hive. The relative abundance of each compound may be at least 10 times, 20 time, 50 times, 100 times, 500 times, 1000 times, 2000 times or 3000 times more in an infected hive than in a non-infected hive. The compound may not be present in a non-infected hive but is present in a hive infected with AFB.

The detection means of the present invention are designed to be particularly sensitive and selective to at least one VOC indicative of AFB infection in a hive. In some aspects and embodiments, the present invention provides a detection means for diagnosing AFB bacteria in a hive, wherein the detection means is configured to detect the presence of at least one VOC indicative of AFB bacteria in a sample thereby affording AFB diagnosis.

Compounds

TABLE 1
VOCs detected in honey bee hives infected with AFB

| | AFB Av. | Healthy Av. | | |
|---|---|---|---|---|
| Compound | Abundance | Abundance | Fold change | Chemical class |
| 1-Decene | 18849 | 0 | infinitive | alkene |
| 2(3H)-Furanone, dihydro-3-hydroxy-4,4- | 191143 | 0 | infinitive | lactone |
| dimethyl- | | | | |
| 2,3-Pentanedione | 59140 | 2705 | 22 | ketone |
| 2-Coumaranone | 78084 | 0 | infinitive | lactone |
| 2-Dodecanone | 23468 | 0 | infinitive | ketone |
| 2-Pentadecanone | 48077 | 4175 | 12 | ketone |
| 2-Piperidinone | 45783 | 0 | infinitive | lactam |
| 2-Tetradecanone | 36722 | 112 | 328 | ketone |
| 2-Tridecanone | 399402 | 9426 | 42 | ketone |
| 2-Undecanone | 266426 | 1522 | 175 | ketone |
| 3-Buten-1-ol, 3-methyl- | 716858 | 197894 | 4 | alcohol |
| 3-Pentanone, 2,4-dimethyl- | 69384 | 0 | infinitive | ketone |
| 4-Hexen-3-ol | 37553 | 0 | infinitive | alcohol |
| Acetamide | 63921 | 0 | infinitive | amide |
| AFB NIL 1 | 412822 | 36627 | 11 | |
| AFB NIL 2 | 571629 | 30200 | 19 | |
| AFB NIL 6 | 4711294 | 5222 | 902 | |
| AFB NIL 7 | 1242314 | 394 | 3154 | |
| AFB NIL 8//C16 alkene | 16288 | 0 | infinitive | alkene |
| AFB NIL 9 | 131672 | 0 | infinitive | |
| Benzeneacetamide | 354252 | 4288 | 83 | amide |
| Butanal, 2-methyl- | 132299 | 2673 | 49 | aldehyde |
| Butanal, 3-methyl- | 83736 | 3842 | 22 | aldehyde |
| Butanamide | 1198624 | 2363 | 507 | amide |
| Butanamide, 3-methyl- | 6996647 | 11599 | 603 | amide |
| C21 Ketone | 149259 | 0 | infinitive | ketone |
| Trisulfide, dimethyl- | 986739 | 0 | infinitive | sulfur compound |
| Disulfide, dimethyl- | 310361 | 0 | infinitive | sulfur compound |
| Hexanamide | 849630 | 2205 | 385 | amide |
| Indole | 89341 | 0 | infinitive | heteroaromatics |
| N-(3-Methylbutyl)acetamide | 27805 | 0 | infinitive | amide |
| Pentanamide, 4-methyl- | 289760 | 0 | infinitive | amide |
| Pentanoic acid, 3-methyl-2-oxo-, methyl | 370138 | 0 | infinitive | ester |
| ester | | | | |
| Propanamide, 2-methyl- | 1001525 | 734 | 1365 | amide |
| Pyrazine, 2,5-dimethyl- | 8697548 | 0 | infinitive | heteroaromatics |
| Pyrazine, trimethyl- | 293254 | 0 | infinitive | heteroaromatics |
| Furan, 3-methyl- | 696410 | 9688 | 71 | heteroaromatics |

Preferably, the compound used to detect AFB in honey bee hives is present in at least three times the amount present in an uninfected hive. Such compounds may be chosen from the following: 1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS # 3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS # 123-32-0); Pyrazine, trimethyl-(CAS #14667-55-1); Propanamide, 2-methyl-(CAS #563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS #112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS #590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ C16 alkene; AFB NIL 9; Furan, 3-methyl-(CAS #930-27-8).

Preferably, the compounds are chosen from the list comprising: 2(3H)-Furanone, dihydro-3-hydroxy-4,4-dimethyl-(CAS # 52126-90-6); 3-Buten-1-ol, 3-methyl-(CAS #763-32-6); Acetamide (CAS #60-35-5); Benzeneacetamide (CAS #103-81-1); Butanamide (CAS #541-35-5); Butanamide, 3-methyl-(CAS #541-46-8); Hexanamide (CAS #628-02-4); Pentanoic acid, 3-methyl-2-oxo-, methyl ester (CAS #3682-42-6); Propanamide, 2-methyl-(CAS #563-83-7); Pyrazine, 2,5-dimethyl-(CAS #123-32-0); AFB NIL 1; AFB NIL 2; AFB NIL 6; AFB NIL 7.

Preferably, the compound used to detect AFB in honey beehives is present in at least ten times the amount present in an uninfected hive. Such compounds may be chosen from the following: 1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS # 3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS # 123-32-0); Pyrazine, trimethyl-(CAS #14667-55-1); Propanamide, 2-methyl-(CAS #563-83-7); Butanamide, 3-methyl-(CAS #541-46-8); Butanamide (CAS #541-35-5); Hexanamide (CAS # 628-02-4); 2-Tetradecanone (CAS #2345-27-9); 2-Undecanone (CAS #112-12-9); Benzeneacetamide (CAS #103-81-1); Butanal, 2-methyl-(CAS #96-17-3); 2-Tridecanone (CAS # 593-08-8); 2,3-Pentanedione (CAS #600-14-6); Butanal, 3-methyl-(CAS #590-86-3); AFB NIL 2; 2-Pentadecanone (CAS #2345-28-0); AFB NIL 1; AFB NIL 6; AFB NIL 7; AFB NIL 8\ C16 alkene; AFB NIL 9; Furan, 3-methyl-(CAS #930-27-8).

Preferably, the compound used to detect AFB in honey beehives is not present in uninfected hives in detectable levels. Such compounds may be chosen from the following: 1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #624-92-0); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS # 14667-55-1); AFB NIL 8\ C16 alkene; AFB NIL 9.

Method for detection

The present invention provides a method for detecting the presence of American foulbrood (AFB) in a beehive, said method comprising the step of:
a) taking a sample from the beehive;
b) testing the sample for compounds from the list comprising:
1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS # 123-32-0); Pyrazine, trimethyl-(CAS #14667-55-1); Propanamide, 2-methyl-(CAS # 563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS #112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS # 590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ 016 alkene; AFB NIL 9; Furan, 3-methyl-(CAS #930-27-8)
   wherein the presence of one or more of the compounds in a relative amount of at least two times that of a non-infected hive indicates the presence of AFB in the beehive.

Preferably, the compounds are chosen from the list comprising: 2(3H)-Furanone, dihydro-3-hydroxy-4,4-dimethyl-(CAS #52126-90-6); 3-Buten-1-ol, 3-methyl-(CAS #763-32-6); Acetamide (CAS #60-35-5); Benzeneacetamide (CAS #103-81-1); Butanamide (CAS #541-35-5); Butanamide, 3-methyl-(CAS #541-46-8); Hexanamide (CAS #628-02-4); Pentanoic acid, 3-methyl-2-oxo-, methyl ester (CAS #3682-42-6); Propanamide, 2-methyl-(CAS #563-83-7); Pyrazine, 2,5-dimethyl-(CAS #123-32-0); AFB NIL 1; AFB NIL 2; AFB NIL 6; AFB NIL 7.

The compounds are preferably volatile organic compounds (VOC). VOCs are organic chemicals that have a high vapour pressure at ordinary room temperature. The term “volatile organic compounds (VOC)” refers to organic compounds having an initial boiling point less than or equal to 250 ° C. (482 ° F.) measured at a standard atmospheric pressure of 101.3 kPa.

Preferably, method of the present invention detects the compounds provided in Table 1 in an AFB infected hive in amounts of at least two times that present in a non-infected hive. The relative abundance of each compound may be at least 2 times, 3 times, 4 times, 5 times, 10 times, 20 time, 50 times, 100 times, 500 times, 1000 times, 2000 times or 3000 times more in an infected hive than in a non-infected hive. The compound may not be present in detectable levels a non-infected hive but is present in a hive infected with AFB.

Preferably, the method of the present invention detects the presence of at least one compound, more preferably two compounds from those provided in Table 1. However, the testing may detect three, four, five or more compounds. The degree of certainty of detection of AFB in a beehive increases with the number of compounds from Table 1 detected.

The method of the present invention may detect both one or more compound from Table 1 (i.e. at least one, two, three, four, five or more compounds) and one or more of those compounds may be detected at levels at least 2 times, 3 times, 4 times, 5 times, 10 times, 20 time, 50 times, 100 times, 500 times, 1000 times, 2000 times etc more in an infected hive than in a non-infected hive. It is to be understood that the more compounds and the greater the difference between the levels of those compounds in healthy versus infected hives, the more accurate the detection test.

Sample

Preferably the sample is a sample of gas from the interior of the beehive. Preferably, the compounds of the invention are measured inside the entrance/door of the hive. This approach offers the advantage that hives can remain closed and undisturbed throughout disease assessment, preventing disease spread and offering early and fast, non-invasive diagnostics.

Alternatively, the sample may be a sample of tissue (for example tissue from a pupa, larva or a mature bee) or a sample of honey or wax from the hive. Bee debris may also be used as a sample. The debris is small particles of wax that form when bees uncover stored glycide stores in the honeycombs during the wintertime; the bee debris (including propolis, body parts, etc.) falls to a mat on the floor of the hive where this debris can be collected.

In certain embodiments, the organic compound(s) are detected in the gas phase. The sample itself may be in the gas phase, for example air in the headspace of the hive, or the gas may be mixed with or generated from a solid or liquid sample, such as a sample of tissue.

In certain embodiments, a detector determines the presence or absence, or alternatively the concentration, of the headspace VOCs in the gas phase to determine whether there is an active AFB bacterial infection in the hive. Accordingly, the sample container is designed to prohibit the release of gas from the sample container or the introduction of ambient gas into the sample container.

The samples may be taken repeatedly throughout the peak honey making season and/or at honey harvest time. Generally, samples would be taken and tested starting late winter, and continuing during spring and into summer. Additionally, samples may be taken and tested when hives are moved and/or new hives introduced to an apiary or pollination site.

The sample may be taken using a detecting method that utilises a handheld or mobile detection means. Alternatively, the sample may be taken using a detecting method that utilises a detection means placed permanently or semi-permanently within the hive. For example, the detection means may be placed in the headspace of the hive in a permanent or semi-permanent manner.

The present invention further provides a device for detecting the presence of American foulbrood (AFB) in a beehive, said device comprising:
i.) a means for testing the sample for compounds from the list comprising:
   1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS #14667-55-1); Propanamide, 2-methyl-(CAS # 563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS # 112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS # 590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ C16 alkene; AFB NIL 9; Furan, 3-methyl-(CAS #930-27-8)
      wherein the presence of one or more of the compounds in a relative amount of at least three times that of a non-infected hive indicates the presence of AFB in the beehive.

The one or more VOCs may be detected using various technologies including, but not limited to: gas chromatography (GC); spectrometry, for example mass spectrometry (including quadrupole, time of flight, tandem mass spectrometry, ion cyclotron resonance, and/or sector (magnetic and/or electrostatic)), ion mobility spectrometry, field asymmetric ion mobility spectrometry, and/or DMS; fuel cell electrodes; light absorption spectroscopy; Raman spectroscopy; nanoparticle technology; flexural plate wave (FPW) sensors; biosensors that mimic naturally occurring cellular mechanisms; acoustic sensors: quartz crystal microbalance or surface acoustic wave sensors; resistance based sensors: metallic resistors, semiconducting metal oxide sensors; work function based sensors: metal-semiconductor (Schottky) diode, metal-insulator-semiconductor transistors, and metal-insulator-semiconductor capacitors; thermal conductivity sensors; catalytic sensors: pellistors and thermoelectric; electrochemical sensors; photoacoustic equipment; laser-based equipment; electronic noses (bio-derived, surface coated); microelectromechanical system (MEMS); pulsed discharge ionization detector (PDID); various ionization techniques; aptamer based lateral flow strips; antibody-based screening strips; colorimetric assays and/or trained animal detection. The detection method may be combined with a pre-concentrator (PC), including micro-PC and Solid-Phase Microextraction (SPME) to determine the presence of compounds associated with AFB infections.

Preferably the detection means is a handheld, mobile, portable or point-of-use diagnostic tool. The detection means may be single use, or rechargeable and able to be used multiple times. Alternatively, the detection means may be a laboratory based diagnostic tool.

In certain embodiments, a point-of-use diagnostic tool is used to identify bacterial infection VOC biomarkers. A point-of-care diagnostic tool, such as a micromachined Differential Mobility Spectrometer (“DMS”), preferably is portable and may detect VOCs to low limits of detection. Accordingly, in certain embodiments, the present invention includes a library of VOC data and relevant information for a point-of-care diagnostic tool that may be used to identify bacteria in a sample, or the presence of a bacterial infection obtained from one or more sources.

The samples may be tested at the location of the beehive or taken away to another location for testing to determine of compounds associated with AFB are present.

A wide range of methods and means for detecting compounds, both in solid or liquid tissue samples, or in gas samples such as air from the interior of the beehive. The skilled reader will understand and be able to choose a suitable detection method and means for the compounds provided in the present invention that are indicative of infection by AFB.

The relative abundance of each compound may be at least 2 times, 3 times, 4 times, 5 times, 10 times, 20 time, 50 times, 100 times, 500 times, 1000 times, 2000 times or 3000 times more in an infected hive than in a non-infected hive. The compound may not be present in a non-infected hive but is present in a hive infected with AFB.

Preferably, the method of the present invention detects the presence of at least one, two, three, four, five or more compounds from those provided in Table 1.

Preferably the sample is a sample of gas from the interior of the beehive. Alternatively, the sample may be a sample of tissue, honey, wax or bee debris from the hive.

Solid phase microextraction and gas chromatography (SPME GC-MS)

Gas chromatography—mass spectrometry (GC-MS) is an example of a detection means that may be used, utilising SPME fibres as the means of VOC absorption, and a WAX column as the means to separate the compounds inside the GC before detection in the mass spectrometer (MS). The total ion chromatogram (TIC) is initially used to detect compounds only present in the samples of AFB-infected brood.

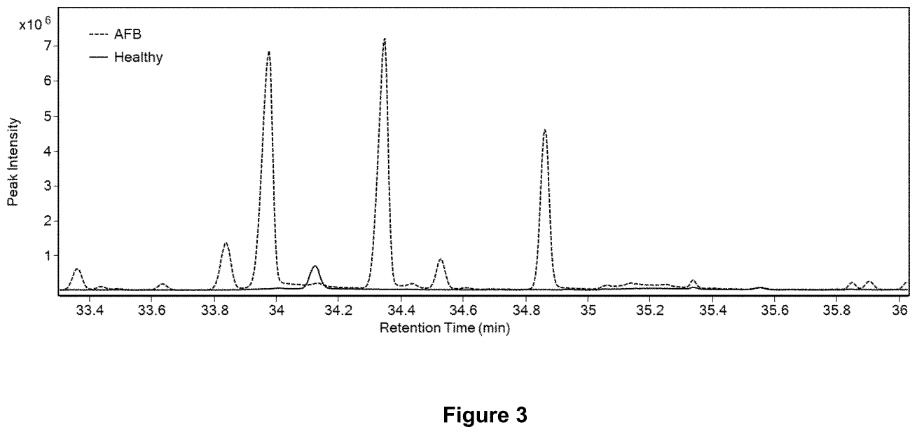

It is often not possible to determine relevant VOC peaks for the detection of AFB in a sample based on peak height or % of TIC alone, as there are other volatiles that are very prominent in the GC-MS chromatogram, for example the volatiles emitted from wood, honey or pollen. For this purpose, compounds that are only present in hives with AFB infections but generally absent from healthy hives are selected (FIG. 3). Furthermore, as there are generally more healthy bees in a hive than AFB infected larvae, there may be high levels of the VOCs associated with healthy bees. VOCs are also emitted from hive material such as honey, pollen and wax, as well as the material the hive is built from such as wood.

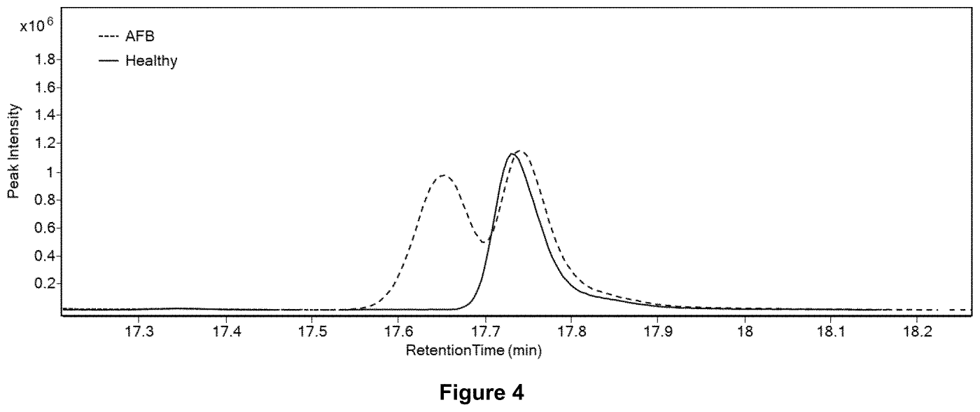

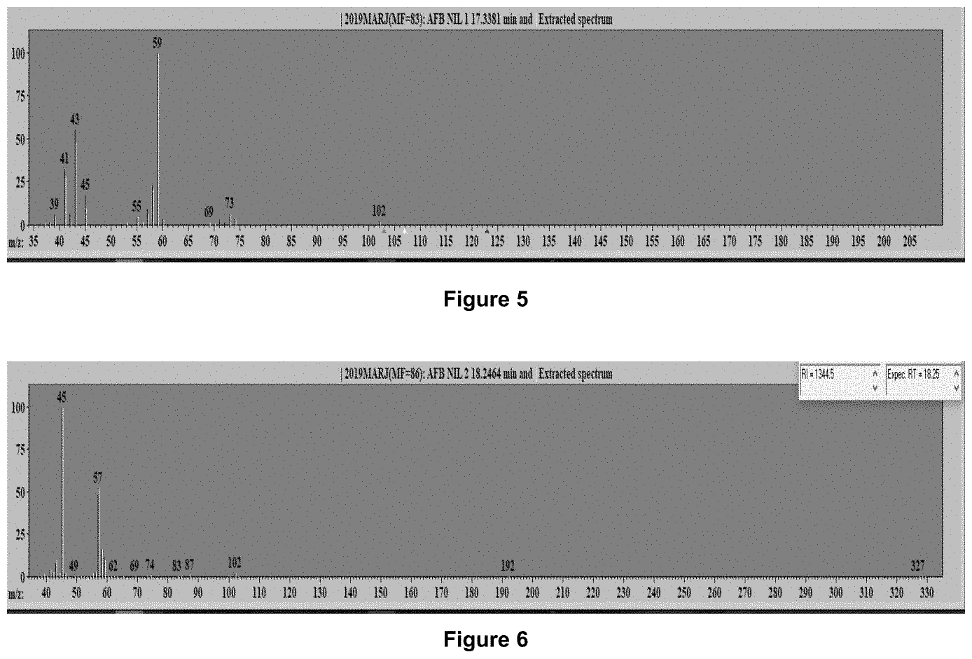

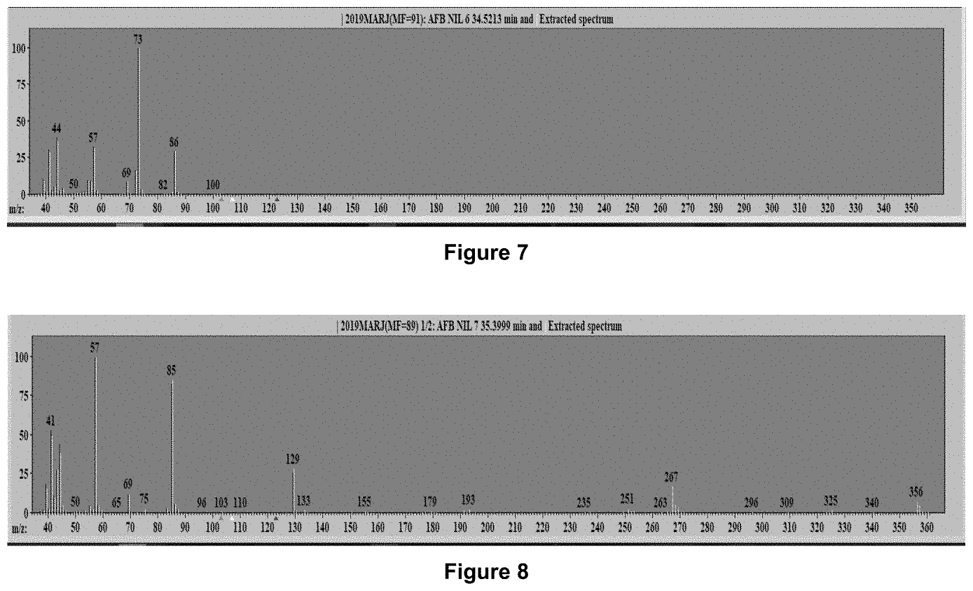

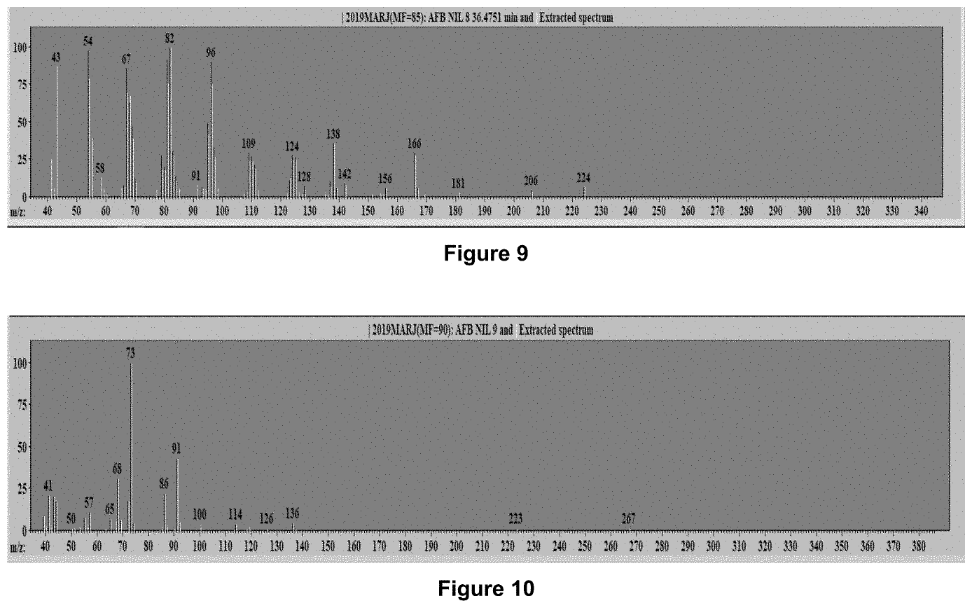

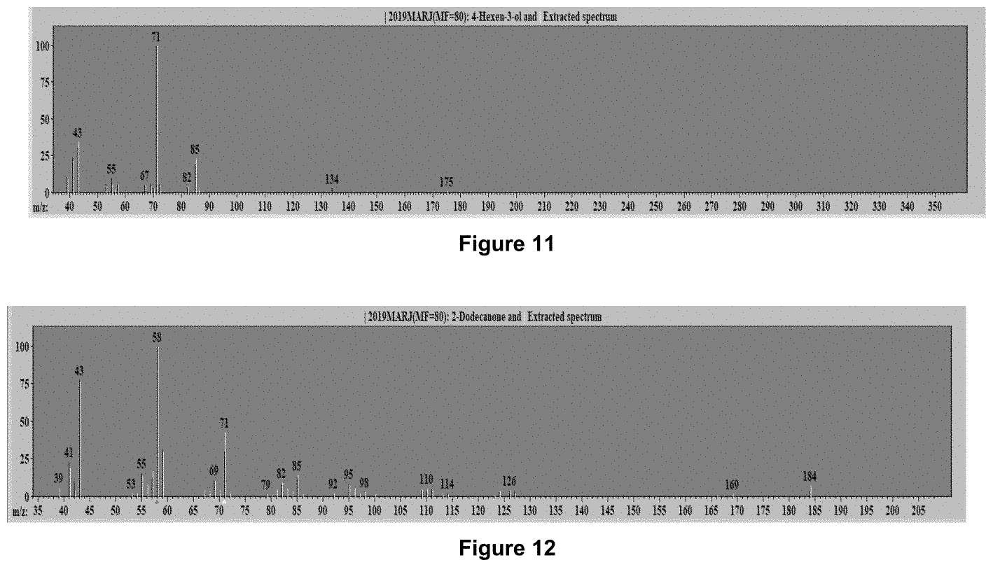

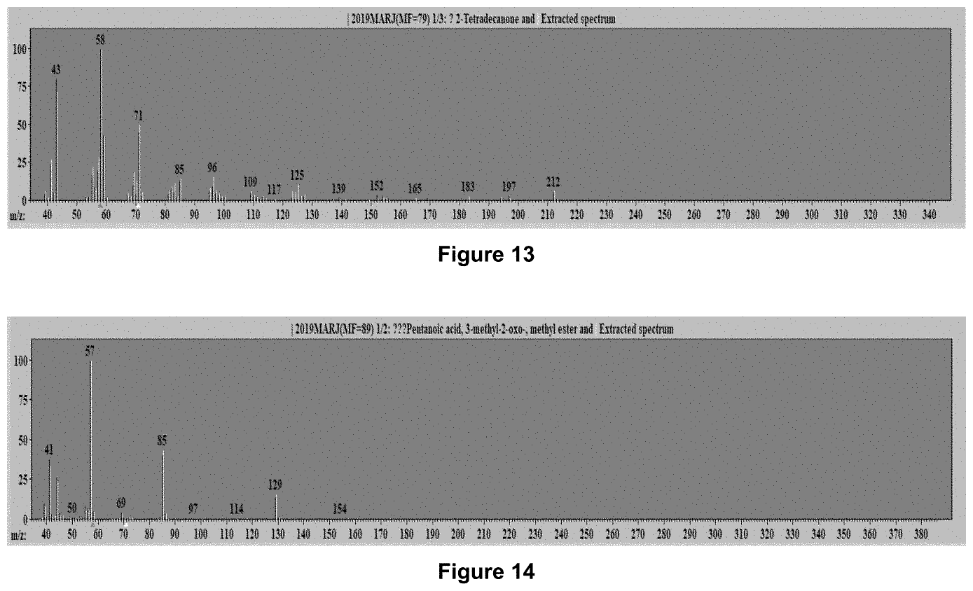

When reviewing the TIC, the following features are used to select peaks for review to identify VOC indicative of the presence of AFB (FIG. 4):
shape: uniform smooth peak (deconvolution can be used to smooth shoulders or extra bumps);
signal to noise ratio: at least 30, more preferably at least 40, most preferably at least 50 (it has been noted that peaks with S/N ratios less than 50 are hard to quantitate and have low confidence identifications).

Preferably, the VOCs used to detect the presence of AFB in a sample have a TIC minimum peak intensity of at least 5000 in 80% of the affected samples. More preferably the VOCs used to detect the presence of AFB in a sample have a TIC minimum peak intensity of at least 5000 in 100% of the samples taken from a single hive. The VOCs detected may be those produced by AFB and those present in healthy, non-infected hives.

Kits

The present invention provides a kit for detecting the presence of American foulbrood (AFB) in a beehive, said kit comprising:
i.) a means for testing the sample for compounds from the list comprising:
   1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS #14667-55-1); Propanamide, 2-methyl-(CAS #563-83-7); Butanamide, 3-methyl-(CAS # 541-46-8); Butanamide (CAS #541-35-5); Hexanamide (CAS #628-02-4); 2-Tetradecanone (CAS #2345-27-9); 2-Undecanone (CAS #112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS #590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ C16 alkene; AFB NIL 9; Furan, 3-methyl-(CAS #930-27-8); and
ii.) instructions for use
   wherein the presence of one or more of the compounds in a relative amount of at least three times that of a non-infected hive indicates the presence of AFB in the beehive.

The kit may further comprise a means for taking a sample from the beehive. For example, the kit may contain a suction device and container for sampling air from the inside of the hive.

Device

The present invention further provides a device for detecting the presence of American foulbrood (AFB) in a beehive, said device comprising:
i.) a means for testing the sample for compounds from the list comprising:
   1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS #14667-55-1); Propanamide, 2-methyl-(CAS # 563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS # 112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS #590-86-3); AFB NIL 2; 2-Pentadecanone (CAS #2345-28-0); AFB NIL 1; 3-Buten-1-ol, 3-methyl-(CAS #763-32-6); AFB NIL 6; AFB NIL 7; AFB NIL 8\ 016 alkene; AFB NIL 9; Furan, 3-methyl-(CAS #930-27-8)
      wherein the presence of one or more of the compounds in a relative amount of at least three times that of a non-infected hive indicates the presence of AFB in the beehive.

The means for testing for one or more VOCs may be selected from various technologies including, but not limited to: gas chromatography (GC); spectrometry, for example mass spectrometry (including quadrupole, time of flight, tandem mass spectrometry, ion cyclotron resonance, and/or sector (magnetic and/or electrostatic)), ion mobility spectrometry, field asymmetric ion mobility spectrometry, and/or DMS; fuel cell electrodes; light absorption spectroscopy; Raman spectroscopy; nanoparticle technology; flexural plate wave (FPW) sensors; biosensors that mimic naturally occurring cellular mechanisms; acoustic sensors: quartz crystal microbalance or surface acoustic wave sensors; resistance based sensors: metallic resistors, semiconducting metal oxide sensors; work function based sensors: metal-semiconductor (Schottky) diode, metal-insulator-semiconductor transistors, and metal-insulator-semiconductor capacitors; thermal conductivity sensors; catalytic sensors: pellistors and thermoelectric; electrochemical sensors; photoacoustic equipment; laser-based equipment; electronic noses (bio-derived, surface coated); microelectromechanical system (MEMS); pulsed discharge ionization detector (PDID); various ionization techniques; aptamer based lateral flow strips; antibody-based screening strips; colorimetric assays; and/or trained animal detection. The detection method may be combined with a pre-concentrator (PC), including micro-PC and Solid-Phase Microextraction (SPME) to determine the presence of compounds associated with AFB infections.

Preferably the detection means is a handheld, mobile, portable or point-of-use diagnostic tool. The detection means may be single use, or rechargeable and able to be used multiple times. Alternatively, the detection means may be a laboratory based diagnostic tool.

In certain embodiments, a point-of-use diagnostic tool is used to identify bacterial infection VOC biomarkers. A point-of-care diagnostic tool, such as a micromachined Differential Mobility Spectrometer (“DMS”), preferably is portable and may detect VOCs to low limits of detection. Accordingly, in certain embodiments, the present invention includes a library of VOC data and relevant information for a point-of-care diagnostic tool that may be used to identify bacteria in a sample, or the presence of a bacterial infection obtained from one or more sources.

For the device or kit of the present invention, preferably the compounds identified are chosen from the list comprising: 2(3H)-Furanone, dihydro-3-hydroxy-4,4-dimethyl-(CAS #52126-90-6); 3-Buten-1-ol, 3-methyl-(CAS #763-32-6); Acetamide (CAS #60-35-5); Benzeneacetamide (CAS #103-81-1); Butanamide (CAS #541-35-5); Butanamide, 3-methyl-(CAS #541-46-8); Hexanamide (CAS #628-02-4); Pentanoic acid, 3-methyl-2-oxo-, methyl ester (CAS #3682-42-6); Propanamide, 2-methyl-(CAS #563-83-7); Pyrazine, 2,5-dimethyl-(CAS #123-32-0); AFB NIL 1; AFB NIL 2; AFB NIL 6; AFB NIL 7.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1: Testing for AFB from Infected Brood Samples

Methods

### Sample Collection

Beehive brood frames suspected of having American foulbrood (n=8) were received either directly from the beekeeper or via the Department of Primary Industries and Rural Development (DPIRD) Western Australia. Healthy brood frames (n=6) were sourced from the UWA Apiary. Samples were stored at -20° C. for PCR and volatile analyses.

### AFB Confirmation

American foulbrood infection was confirmed using established PCR methods. DNA was extracted directly from two larval remains following an established protocol (Dobbelaere, de Graaf & Peeters 2001). Briefly, the remains of two larvae displaying symptoms of AFB (i.e. a ropy mass or dried scale) were suspended in 1 ml ddH₂O and thoroughly vortexed. 100 μl of the suspension was heated at 95° C. for 15 min at 1000 rpm. After cooling on ice for 5 min, the suspension was centrifuged at 5000 rpm for 5 min. The supernatant was then used as template DNA in PCR reactions. DNA was extracted from two larvae from each healthy brood frame in the same manner.

PCR was carried out on samples of AFB-suspected larvae and healthy brood using primers developed for AFB identification by Dainat et al. (2018) (Table 1). The reaction mixture (50 μl) contained 5 μl PCR buffer (Labtaq), 1.25 μl polymerase (Labtaq), 1.25 μl of both forward and reverse primer, 200 μM of each dNTP (New England Biolabs) and 3 μl of template DNA. Amplification was performed in a T100™ Thermal Cycler (Bio-Rad) and consisted of an initial activation of the polymerase at 95° C. for 10 min, followed by 30 cycles of: denaturation at 93° C. for 15 s; annealing at 55° C. for 30 s; extension at 72° C. for 30 s; ending with a final extension at 72° C. for 7 min. Samples (3 μl) of amplified products were electrophoresed in 2% agarose in Tris-borate-EDTA buffer containing ethidium bromide (0.5 μg.m1⁻¹). The amplimers were visualised on a UV transilluminator and photographed using the Bio-Rad Molecular Imager Gel Doc XR+.

TABLE 2
Primer sequences used and theoretical length of
amplicon.

| SEQ | | | | |
|---|---|---|---|---|
| ID | | | Product | |
| NO | Primer | Sequence (5′ to 3′) | (bp) | Reference |
| 1 | PL-F | TACGCTTTTCGATTCTCTG | 87 | Dainat et al. |
| 2 | PL-R | GTCTGTACTGAACCAAGTC | | 2018 |

All AFB-suspected samples showed positive PCR results for P. larvae. P. larvae was not detected in healthy brood samples.

### Headspace Volatile Sampling

Headspace sampling was achieved using solid phase microextraction (HS-SPME). Approximately 1 g of a sample's brood (approximately 20-30 larval remains) were placed in a 20 ml headspace glass vial (Agilent Technologies) and heated at 50° C. for 10 minutes. Between three and ten SPME-samples were made from each frame of AFB material (dependent on the number of larval remains available). The septum covering the vial headspace was pierced with the SPME needle and then the fibre was protracted. The fibre was exposed to the headspace for 10 minutes at 50° C. SPME Fibre, 50/30um DVB/Carboxen/Polydimethylsiloxane (DVB/CAR/PDMS) (Supelco). The extracted analytes desorbed in the injection port of the GC-MS.

Analysis of volatiles by GC-MS

Analyses were performed with an Agilent Technologies 7890A gas chromatograph coupled to a quadrupole mass spectrometer 5975C inert XL MSD with Triple-Axis Detector. The GC system was equipped with a fused silica capillary column (DB-WAX UI).

Splitless thermal desorption was performed at 250° C. and used a solvent delay of 1 min. Helium was used as the carrier gas with a flow rate was 1 ml/min. The oven temperature was programmed as follows: initial temperature 40° C., hold for 2 min, then increase to 80° C. at a rate of 3° C./min, hold for 3 min, then increase to 150° C. at a rate of 5° C./min, then to 250° C. at 10° C./min with 10 min of final isotherm. The transfer line temperature was kept at 250° C., and the ion source temperature was 230° C. The detector operated in scan mode from 35 to 400 Da with a scanning velocity of 3.89 scans/s.

Volatile compounds in the collected spectra were identified by first deconvoluting spectral peaks with the Automated Mass Spectral Deconvolution and Identification System (AMDIS, Version 2.64, 2006) and then comparing mass spectra and GC retention times to the National Institute of Standards and Technologies (NIST) Mass Spectral Library with NIST Mass Spectral Search Program (NIST/EPA/NIH/ Mass Spectral Library, Version 2.0.d, 2005). Agilent MassHunter Qualitative Analysis for GCMS (Agilent Technologies, Version B.07.01 2008) software was used to quantitate peak intensities.

### Statistical Analysis

Averages and fold changes for each treatment were calculated in Excel. The t.test function was used to calculate statistical significance of group mean differences. Further statistical analyses were performed in RStudio version 1.1.447. Integrated peak intensities of the compounds, found by HS-SPME-GC-MS analysis, were organised into a matrix, with rows corresponding to samples and columns corresponding to the identified chemical compound. Principal components analysis (PCA) was performed on the raw data using the package ggplot version 3.2.0. Data were log10 transformed and scaled from 0 to 8 prior to heatmap analysis using the package ggplots version 3.0.1.1. Heatmap dendrograms were created using the complete linkage method for hierarchical clustering. Compound identities were confirmed with commercial standards where possible.

Results

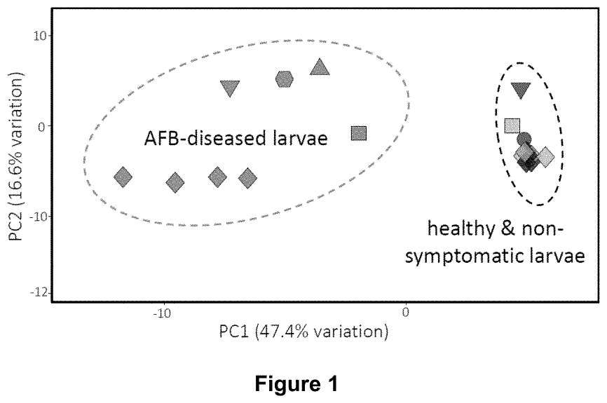

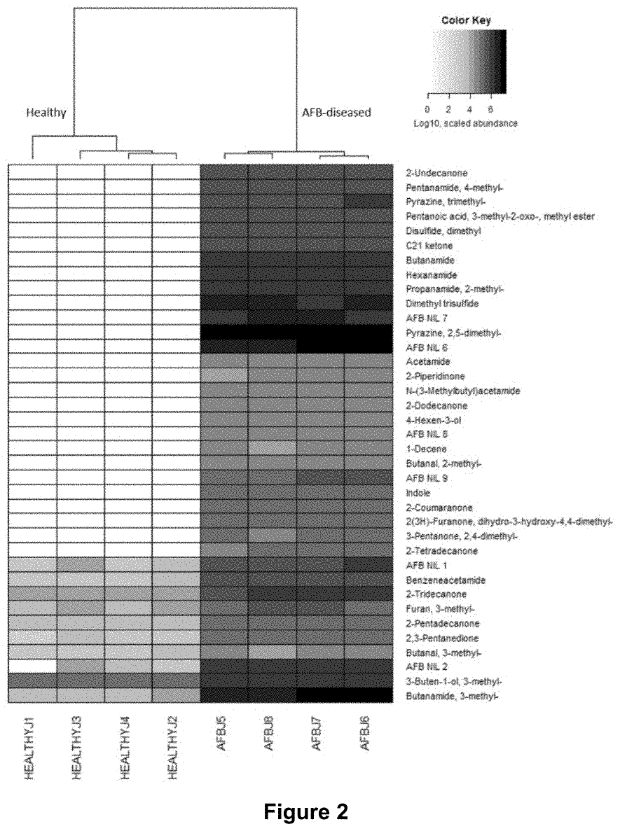

A total of 116 chemical compounds were detected across 65 samples taken from 6 healthy hives and 8 hives with AFB infections. Average compound peak intensity and fold-changes between treatments indicated that compounds occur in significantly higher abundances in AFB diseased larvae (Table 3). PCA analysis shows the volatile profiles are significantly different between healthy and advanced-AFB diseased larvae (FIG. 1). Heatmap analysis further revealed which compounds are driving these distinct volatile profiles (FIG. 2).

TABLE 3
Chemical compounds detected in honey bee larvae showing average peak
intensity and fold changes between healthy and advanced-AFB diseased
larvae. The data from 3 independent experiments are displayed.

| | | Experiment 1 | | | | Experiment 2 | |
|---|---|---|---|---|---|---|---|
| | NIST | Healthy | | Exp1 | | Healthy | |
| | R.Match | Av. | AFB Av. | Fold- | Exp1 t- | Av. | AFB Av. |
| Compound | Statistic | Abund | Abund | change | test P | Abund | Abund |
| Furan, 3-methyl- | 929 | 12667.5 | 1206964 | 94.8 | <0.001 | N/A | N/A |
| 1-Decene | 942 | 0 | 19083 | >>> | <0.001 | 0 | 10472 |
| 2(3H)-Furanone, dihydro- | 925 | 0 | 87758 | >>> | 0.001 | 0 | 320137 |
| 3-hydroxy-4,4-dimethyl- | | | | | | | |
| 2,3-Pentanedione | 900 | 0 | 44064 | >>> | 0.006 | 7149 | 45790 |
| 2-Coumaranone | 957 | 0 | 10340 | >>> | <0.001 | 0 | 64719 |
| 2-Dodecanone | 845 | 0 | 14567 | >>> | <0.001 | 0 | 10041 |
| 2-Pentadecanone | 866 | 2032 | 27744 | 13.65 | <0.001 | 2022 | 18935 |
| 2-Piperidinone | 937 | 0 | 25697 | >>> | <0.001 | 0 | 66989 |
| 2-Tetradecanone | 824 | 0 | 19712 | >>> | <0.001 | 0 | 13605 |
| 2-Tridecanone | 960 | 7368 | 218540 | 29.66 | <0.001 | 5730 | 144375 |
| 2-Undecanone | 902 | 0 | 142396 | >>> | 0.004 | 5062 | 90871 |
| 3-Buten-1-ol, 3-methyl- | 948 | 152703 | 864259 | 5.66 | 0.033 | 213135 | 230852 |
| 3-Pentanone, 2,4- | 844 | 0 | 55875 | >>> | <0.001 | 0 | 41524 |
| dimethyl- | | | | | | | |
| 4-Hexen-3-ol | 827 | 0 | 23482 | >>> | <0.001 | 0 | 52397 |
| Acetamide | 974 | 0 | 57509 | >>> | <0.001 | 0 | 89722 |
| AFB NIL 1 | | 1913 | 312642 | 163.43 | 0.002 | 104916 | 280488 |
| AFB NIL 2 | | 0 | 396150 | >>> | 0.001 | 83439 | 373993 |
| AFB NIL 6 | | 16899 | 4112135 | 243.34 | <0.001 | 4052 | 4252810 |
| AFB NIL 7 | | 1596 | 778624 | 488.01 | <0.001 | 699 | 720969 |
| AFB NIL 8//C16 alkene | | 0 | 9878 | >>> | <0.001 | 0 | 7259 |
| AFB NIL 9 | | 0 | 88721 | >>> | <0.001 | 0 | 92531 |
| Benzeneacetamide | 948 | 6218 | 240069 | 38.61 | <0.001 | 3850 | 286441 |
| Butanal, 2-methyl- | 866 | 0 | 44062 | >>> | 0.003 | 8688 | 273992 |
| Butanal, 3-methyl- | 909 | 0 | 40207 | >>> | 0.002 | 7834 | 159285 |
| Butanamide | 901 | 6343 | 910252 | 143.5 | <0.001 | 991 | 1221428 |
| Butanamide, 3-methyl- | 909 | 26248 | 6170695 | 235.1 | <0.001 | 8237 | 7290496 |
| C21 Ketone | | 0 | 85444 | >>> | <0.001 | 0 | 59096 |
| Trisulfide dimethyl | 947 | 0 | 252144 | >>> | 0.001 | 0 | 327335 |
| Disulfide, dimethyl- | 964 | 754 | 268336 | 355.73 | <0.001 | 0 | 267839 |
| Hexanamide | 929 | 6834 | 599198 | 87.67 | <0.001 | 2564 | 698929 |
| Indole | 913 | 0 | 40958 | >>> | <0.001 | 0 | 40008 |
| N-(3- | 903 | 0 | 18323 | >>> | <0.001 | 0 | 18554 |
| Methylbutyl)acetamide | | | | | | | |
| Pentanamide, 4-methyl- | 899 | 0 | 187446 | >>> | <0.001 | 0 | 187541 |
| Pentanoic acid, 3-methyl- | 840 | 0 | 283204 | >>> | <0.001 | 0 | 227012 |
| 2-oxo-, methyl ester | | | | | | | |
| Propanamide, 2-methyl- | 906 | 3179 | 878849 | 276.44 | <0.001 | 0 | 967834 |
| Pyrazine, 2,5-dimethyl- | 909 | 0 | 6499214 | >>> | 0.027 | 0 | 5084145 |
| Pyrazine, trimethyl- | 934 | 0 | 165047 | >>> | 0.014 | 0 | 188750 |

| | Experiment 2 | | Experiment 3 | | | |
|---|---|---|---|---|---|---|
| | Exp2 | | Healthy | | Exp3 | |
| | Fold- | Exp2 t- | Av. | AFB Av. | Fold- | Exp3 t- |
| Compound | change | test P | Abund | Abund | change | test P |
| Furan, 3-methyl- | N/A | NA | 8197.5 | 278683.9 | 34 | 0.007 |
| 1-Decene | >>> | 0.001 | 0 | 27931 | >>> | <0.001 |
| 2(3H)-Furanone, dihydro- | >>> | 0.004 | 0 | 132312 | >>> | <0.001 |
| 3-hydroxy-4,4-dimethyl- | | | | | | |
| 2,3-Pentanedione | 6.4 | 0.004 | 2105 | 88498 | 42 | <0.001 |
| 2-Coumaranone | >>> | <0.001 | 0 | 148092 | >>> | <0.001 |
| 2-Dodecanone | >>> | <0.001 | 0 | 45410 | >>> | <0.001 |
| 2-Pentadecanone | 9.37 | <0.001 | 7524 | 96117 | 12.8 | <0.001 |
| 2-Piperidinone | >>> | <0.001 | 0 | 39084 | >>> | <0.001 |
| 2-Tetradecanone | >>> | <0.001 | 0 | 75803 | >>> | <0.001 |
| 2-Tridecanone | 25.2 | <0.001 | 12823 | 824283 | 64.3 | <0.001 |
| 2-Undecanone | 17.95 | 0.002 | 0 | 559356 | >>> | <0.001 |
| 3-Buten-1-ol, 3-methyl- | 1.08 | 0.47 | 211247 | 1126447 | 5.3 | <0.001 |
| 3-Pentanone, 2,4- | >>> | <0.001 | 0 | 111624 | >>> | <0.001 |
| dimethyl- | | | | | | |
| 4-Hexen-3-ol | >>> | 0.001 | 0 | 33127 | >>> | <0.001 |
| Acetamide | >>> | <0.001 | 0 | 40337 | >>> | <0.001 |
| AFB NIL 1 | 2.67 | 0.105 | 8459 | 639152 | 75.6 | <0.001 |
| AFB NIL 2 | 4.48 | 0.007 | 9808 | 930806 | 94.9 | <0.001 |
| AFB NIL 6 | 1049.69 | <0.001 | 0 | 5669237 | >>> | <0.001 |
| AFB NIL 7 | 1031.8 | 0.001 | 0 | 2190437 | >>> | <0.001 |
| AFB NIL 8//C16 alkene | >>> | <0.001 | 0 | 31383 | >>> | <0.001 |
| AFB NIL 9 | >>> | <0.001 | 0 | 209200 | >>> | <0.001 |
| Benzeneacetamide | 74.4 | <0.001 | 3603 | 521650 | 144.8 | <0.001 |
| Butanal, 2-methyl- | 31.54 | 0.014 | 0 | 49920 | >>> | <0.001 |
| Butanal, 3-methyl- | 20.33 | 0.009 | 2947 | 36869 | 12.5 | <0.001 |
| Butanamide | 1232.52 | <0.001 | 0 | 1409687 | >>> | <0.001 |
| Butanamide, 3-methyl- | 885.1 | <0.001 | 6179 | 7351864 | 1189.8 | <0.001 |
| C21 Ketone | >>> | <0.001 | 0 | 299830 | >>> | <0.001 |
| Trisulfide dimethyl | >>> | <0.001 | 0 | 2307335 | >>> | <0.001 |
| Disulfide, dimethyl- | >>> | <0.001 | 0 | 391134 | >>> | <0.001 |
| Hexanamide | 272.59 | <0.001 | 0 | 60366 | >>> | <0.001 |
| Indole | >>> | <0.001 | 0 | 182855 | >>> | <0.001 |
| N-(3- | >>> | <0.001 | 0 | 45784 | >>> | <0.001 |
| Methylbutyl)acetamide | | | | | | |
| Pentanamide, 4-methyl- | >>> | <0.001 | 0 | 484983 | >>> | <0.001 |
| Pentanoic acid, 3-methyl- | >>> | <0.001 | 0 | 597192 | >>> | <0.001 |
| 2-oxo-, methyl ester | | | | | | |
| Propanamide, 2-methyl- | >>> | <0.001 | 0 | 1138650 | >>> | <0.001 |
| Pyrazine, 2,5-dimethyl- | >>> | 0.009 | 0 | 14533913 | >>> | <0.001 |
| Pyrazine, trimethyl- | >>> | 0.001 | 0 | 512157 | >>> | <0.001 |

Match Stat (NIST) refers to the raw match statistic from NIST, whereas R.Match Stat, is the NIST match stat using the deconvoluted spectra.

TABLE 4
Characterisation of compounds detected in honey bee larvae

| Compound | CAS number | Formula | RT | MW | Chemical class |
|---|---|---|---|---|---|
| 1-Decene | 872-05-9 | C10H20 | 5.56 | 140 | alkene |
| 2(3H)-Furanone, dihydro-3-hydroxy- | 52126-90-6 | C6H10O3 | 35.909 | 130 | lactone (furanone) |
| 4,4-dimethyl- | | | | | |
| 2,3-Pentanedione | 600-14-6 | C5H8O2 | 5.995 | 100 | ketone |
| 2-Coumaranone | 553-86-6 | C8H6O2 | 36.452 | 134 | lactone |
| 2-Dodecanone | 6175-49-1 | C12H24O | 28.35 | 184 | ketone |
| 2-Pentadecanone | 2345-28-0 | C15H30O | 35.851 | 226 | ketone |
| 2-Piperidinone | 675-20-7 | C5H9NO | 37.275 | 99 | amide (lactam) |
| 2-Tetradecanone | 2345-27-9 | C14H28O | 33.635 | 212 | ketone |
| 2-Tridecanone | 593-08-8 | C13H26O | 32.176 | 198 | ketone |
| 2-Undecanone | 112-12-9 | C11H22O | 26.74 | 170 | ketone |
| 3-Buten-1-ol, 3-methyl- | 763-32-6 | C5H10O | 12.753 | 86 | alcohol |
| 3-Pentanone, 2,4-dimethyl- | 565-80-0 | C7H14O | 16.98 | 114 | ketone |
| 4-Hexen-3-ol | 4798-58-7 | C6H12O | 37.17 | 100 | alcohol |
| Acetamide | 60-35-5 | C2H5NO | 31.141 | 59 | amide |
| AFB NIL 1 | | | 16.249 | 102 | unknown |
| AFB NIL 2 | | | 17.078 | 102 | unknown |
| AFB NIL 6 | | | 33.977 | 100 | unknown |
| AFB NIL 7 | | | 34.864 | 129 | unknown |
| AFB NIL 8//C16 alkene | | | 36.027 | 224 | unknown |
| AFB NIL 9 | | | 36.403 | 267 | unknown |
| Benzeneacetamide | 103-81-1 | C8H9NO | 42.742 | 135 | amide |
| Butanal, 2-methyl- | 96-17-3 | C5H10O | 3.094 | 86 | aldehyde |
| Butanal, 3-methyl- | 590-86-3 | C5H10O | 3.152 | 86 | aldehyde |
| Butanamide | 541-35-5 | C4H9NO | 33.84 | 87 | amide |
| Butanamide, 3-methyl- | 541-46-8 | C5H11NO | 34.347 | 101 | amide |
| C21 Ketone | | | 33.36 | | ketone |
| Trisulfide, dimethyl- | 3658-80-8 | C2H6S3 | 17.646 | 126 | sulfur compound |
| Disulfide, dimethyl- | 624-92-0 | C2H6S2 | 6.229 | 94 | sulfur compound |
| Furan, 3-methyl- | 930-27-8 | C5H6O | 2.872 | 82 | heteroaromatic |
| Hexanamide | 628-02-4 | C6H13NO | 36.977 | 115 | amide |
| Indole | 120-72-9 | C8H7N | 40.368 | 117 | heteroaromatic |
| N-(3-Methylbutyl)acetamide | 13434-12-3 | C7H15NO | 33.438 | 129 | amide |
| Pentanamide, 4-methyl- | 1119-29-5 | C6H13NO | 37.883 | 115 | amide |
| Pentanoic acid, 3-methyl-2-oxo-, | 3682-42-6 | C7H12O3 | 34.529 | 144 | ester |
| methyl ester | | | | | |
| Propanamide, 2-methyl- | 563-83-7 | C4H9NO | 32.145 | 87 | amide |
| Pyrazine, 2,5-dimethyl- | 123-32-0 | C6H8N2 | 15.471 | 108 | heteroaromatic |
| Pyrazine, trimethyl- | 14667-55-1 | C7H10N2 | 19.542 | 122 | heteroaromatic |

### Example 2: Volatile Analysis from In-Hive Samples

Method

Honey bee hives (n=8) were set up with sister queens and n=4 were infected with AFB and monitored weekly. For headspace sampling, each hive was fitted with a spacer above the brood box. The VOCs from the hives were collected through a hole in the spacer located directly above the brood chamber.

### Headspace Volatile Sampling

Headspace sampling was achieved using solid phase microextraction (HS-SPME). The fibre was exposed to the headspace for 30 minutes. SPME Fibre, 50/30 um DVB/Carboxen/Polydimethylsiloxane (DVB/CAR/PDMS) (Supelco). The fibre was retracted and stored in an airtight container. The fibre was then transported back to the lab.

### Analysis of Volatiles by GC-MS

Analyses were performed with an Agilent Technologies 7890A gas chromatograph coupled to a quadrupole mass spectrometer 5975C inert XL MSD with Triple-Axis Detector. The GC system was equipped with a fused silica capillary column (DB-WAX UI).

Splitless thermal desorption was performed at 250° C. and used a solvent delay of 1 min. Helium was used as the carrier gas with a flow rate was 1 ml/min. The oven temperature was programmed as follows: initial temperature 40° C., hold for 2 min, then increase to 80° C. at a rate of 3° C./min, hold for 3 min, then increase to 150° C. at a rate of 5° C./min, then to 250° C. at 10° C./min with 10 min of final isotherm. The transfer line temperature was kept at 250° C., and the ion source temperature was 230° C. The detector operated in scan mode from 35 to 400 Da with a scanning velocity of 3.89 scans/s.

Volatile compounds in the collected spectra were identified by first deconvoluting spectral peaks with the Automated Mass Spectral Deconvolution and Identification System (AMDIS, Version 2.64, 2006) and then comparing mass spectra and GC retention times to the National Institute of Standards and Technologies (NIST) Mass Spectral Library with NIST Mass Spectral Search Program (NIST/EPA/NIH/ Mass Spectral Library, Version 2.0.d, 2005). Agilent MassHunter Qualitative Analysis for GCMS (Agilent Technologies, Version B.07.01 2008) software was used to quantitate peak intensities.

The compounds were compared to the results from the above analysis carried out in vials. This allowed removal of compounds that originated from the wood in the hives, and stored pollen and nectar.

Results

Initial results have shown that a number of biomarkers could distinguish AFB from Control hives in the field.

The compounds identified were: 2(3H)-Furanone, dihydro-3-hydroxy-4,4-dimethyl-(CAS #52126-90-6); 3-Buten-1-ol, 3-methyl-(CAS #763-32-6); Acetamide (CAS #60-35-5); Benzeneacetamide (CAS #103-81-1); Butanamide (CAS #541-35-5); Butanamide, 3-methyl-(CAS #541-46-8); Hexanamide (CAS #628-02-4); Pentanoic acid, 3-methyl-2-oxo-, methyl ester (CAS #3682-42-6); Propanamide, 2-methyl-(CAS #563-83-7); Pyrazine, 2,5-dimethyl-(CAS # 123-32-0); AFB NIL 1; AFB NIL 2; AFB NIL 6; AFB NIL 7.

It is anticipated that the results from the above testing will show a time course of the development of AFB infection, with increasing levels of the compounds identified as being relevant to detection of an AFB infection.

### REFERENCES

Dainat, B, Grossar, D, Ecoffey, B & Haldemann, C 2018, ‘Triplex real-time PCR method for the qualitative detection of European and American foulbrood in honeybee’, Journal of Microbiological Methods, vol. 146, pp. 61-63.

Dobbelaere, W, de Graaf, D C & Peeters, J E 2001, ‘Development of a fast and reliable diagnostic method for American foulbrood disease (Paenibacillus larvae subsp. larvae) using a 16S rRNA gene based PCR’, Apidologie, vol. 32, no. 4, pp. 363-370.

1. A method for detecting the presence of American foulbrood (AFB) in a beehive, said method comprising the step of:
a) taking a sample from the beehive;
b) testing the sample for compounds from the list comprising:
1-Decene (CAS #872-05-9); 2(3H)-Furanone, dihydro-3-hydroxy-4,4-dimethyl-(CAS # 52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS # 1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS #14667-55-1); Propanamide, 2-methyl-(CAS #563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS #112-12-9); Benzeneacetamide (CAS #103-81-1); Butanal, 2-methyl-(CAS # 96-17-3); 2-Tridecanone (CAS #593-08-8); 2,3-Pentanedione (CAS # 600-14-6); Butanal, 3-methyl-(CAS #590-86-3); AFB NIL 2; 2-Pentadecanone (CAS # 2345-28-0); AFB NIL 1; 3-Buten-1-ol, 3-methyl-(CAS #763-32-6); AFB NIL 6; AFB NIL 7; AFB NIL 8\ C16 alkene; AFB NIL 9; Furan, 3-methyl-(CAS #930-27-8)
wherein the presence of one or more of the compounds in a relative amount of at least three times that of a non-infected hive indicates the presence of AFB in the beehive.

2. A kit for detecting the presence of American foulbrood (AFB) in a beehive, said kit comprising:
i.) a means for testing the sample for compounds from the list comprising:
1-Decene (CAS #872-05-9); 2(3H)-Furanone, dihydro-3-hydroxy-4,4-dimethyl-(CAS #52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS #624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS #14667-55-1); Propanamide, 2-methyl-(CAS #563-83-7); Butanamide, 3-methyl-(CAS #541-46-8); Butanamide (CAS # 541-35-5); Hexanamide (CAS #628-02-4); 2-Tetradecanone (CAS #2345-27-9); 2-Undecanone (CAS #112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS #590-86-3); AFB NIL 2; 2-Pentadecanone (CAS #2345-28-0); AFB NIL 1; 3-Buten-1-ol, 3-methyl-(CAS # 763-32-6); AFB NIL 6; AFB NIL 7; AFB NIL 8\ C16 alkene; AFB NIL 9; Furan, 3-methyl-(CAS #930-27-8); and
ii.) instructions for use
wherein the presence of one or more of the compounds in a relative amount of at least three times that of a non-infected hive indicates the presence of AFB in the beehive.

3. A device for detecting the presence of American foulbrood (AFB) in a beehive, said device comprising:
i.) a means for testing the sample for compounds from the list comprising:
1-Decene (CAS #872-05-9); 2(3H)-Furanone, dihydro-3-hydroxy-4,4-dimethyl-(CAS # 52126-90-6); 2-Coumaranone (CAS #553-86-6); 2-Dodecanone (CAS #6175-49-1); 2-Piperidinone (CAS #675-20-7); 3-Pentanone, 2,4-dimethyl-(CAS #565-80-0); 4-Hexen-3-ol (CAS #4798-58-7); Acetamide (CAS #60-35-5); C21 ketone or similar; Trisulfide, dimethyl-(CAS #3658-80-8); Disulfide, dimethyl-(CAS # 624-92-0); Indole (CAS #120-72-9); N-(3-Methylbutyl)acetamide (CAS #13434-12-3); Pentanamide, 4-methyl-(CAS #1119-29-5); Pentanoic acid, 3-methyl-2-oxo-, methyl ester (CAS #3682-42-6); Pyrazine, 2,5-dimethyl-(CAS #123-32-0); Pyrazine, trimethyl-(CAS #14667-55-1); Propanamide, 2-methyl-(CAS # 563-83-7); Butanamide, 3-methyl-(CAS #541-46-8); Butanamide (CAS #541-35-5); Hexanamide (CAS #628-02-4); 2-Tetradecanone (CAS #2345-27-9); 2-Undecanone (CAS #112-12-9); Benzeneacetamide (CAS #103-81-1); Butanal, 2-methyl-(CAS #96-17-3); 2-Tridecanone (CAS #593-08-8); 2,3-Pentanedione (CAS #600-14-6); Butanal, 3-methyl-(CAS #590-86-3); AFB NIL 2; 2-Pentadecanone (CAS #2345-28-0); AFB NIL 1; 3-Buten-1-ol, 3-methyl-(CAS # 763-32-6); AFB NIL 6; AFB NIL 7; AFB NIL 8\ C16 alkene; AFB NIL 9; Furan, 3-methyl-(CAS #930-27-8)
wherein the presence of one or more of the compounds in a relative amount of at least three times that of a non-infected hive indicates the presence of AFB in the beehive.

4. The method of claim 1 wherein one, two, three, four, five, or more of the compounds are present in a relative amount of at least three times that of a non-infected hive.

5. The method of claim 1 wherein the compounds are present in a relative amount of at least 2, 5, 10, 20, 50, 100, 500, 1000, 2000 or 3000 times more in an infected hive than in a non-infected hive.

6. The method of claim 1 wherein the sample is a sample of gas from the interior of the beehive.

7. The method of claim 1 wherein the detection means is a handheld, mobile, portable or point-of-use diagnostic tool.

8. The method of claim 1 wherein the compounds are chosen from the list comprising: 2(3H)-Furanone, dihydro-3-hydroxy-4,4-dimethyl-(CAS #52126-90-6); 3-Buten-1-ol, 3-methyl-(CAS #763-32-6); Acetamide (CAS #60-35-5); B enzeneacetamide (CAS #103-81-1); Butanamide (CAS #541-35-5); Butanamide, 3-methyl-(CAS #541-46-8); Hexanamide (CAS #628-02-4); Pentanoic acid, 3-methyl-2-oxo-, methyl ester (CAS #3682-42-6); Propanamide, 2-methyl-(CAS #563-83-7); Pyrazine, 2,5-dimethyl-(CAS #123-32-0); AFB NIL 1; AFB NIL 2; AFB NIL 6; AFB NIL 7.
